(19) United States
(12) Patent Application Publication
(10) Pub. No.: US 2020/0215223 A1
(43) Pub. Date: Jul. 9, 2020

(54) SURGICAL SEALANT PRODUCTS AND METHOD OF USE

(71) Applicant: St. Teresa Medical, Inc., Eagan, MN (US)

(72) Inventors: Timothy Floyd, Eagle, ID (US); Andy Rock, Eagan, MN (US); Philip A. Messina, Eagan, MN (US); Brian J. Jackson, Eagan, MN (US)

(21) Appl. No.: 16/733,707

(22) Filed: Jan. 3, 2020

Related U.S. Application Data
(60) Provisional application No. 62/787,953, filed on Jan. 3, 2019.

(51) Int. Cl. A61L 24/10; A61B 17/16; A61L 24/00

(52) U.S. Cl. CPC A61L 24/106; A61L 2400/04; A61L 24/0047; A61B 17/1675

(57) ABSTRACT

A surgical joint replacement kit including a first joint replacement component and a surgical sealant. The first joint replacement component is capable of being implanted to replace a joint between a first bone and a second bone. The first bone is cut in conjunction with implanting the first joint replacement component. Cutting the first bone causes a bodily fluid to flow from the first bone. The surgical sealant includes an electrospun dextran base and an effective amount of at least one of fibrinogen and thrombin. The at least one of fibrinogen and thrombin is applied to the electrospun dextran base to form the surgical sealant. The surgical sealant is capable of staunching the flow of the bodily fluid from the first bone.

### REFERENCE TO RELATED APPLICATION

This application claims priority to U.S. Applic. No. 62/787,953, which was filed on Jan. 3, 2019. The contents of which are incorporated herein by reference.

### FIELD OF THE INVENTION

The invention generally relates to products having hemostatic characteristics. More particularly, the invention relates to surgical sealant products and the use thereof.

### BACKGROUND OF THE INVENTION

The body's natural response to stem bleeding from a wound is to initiate blood clotting via a complex process known as the coagulation cascade. The cascade involves two pathways that ultimately lead to the production of the enzyme thrombin, which catalyzes the conversion of fibrinogen to fibrin.

Fibrin is then cross-linked to form a clot, resulting in hemostasis. For wounds that are not severe, and in individuals that have no countervening conditions, the body is usually able to carry out this process efficiently in a manner that prevents excessive loss of blood from the wound. However, in the case of severe wounds, or in individuals in whom the clotting mechanism is compromised, this may not be the case.

For such individuals, it is however possible to administer components of the coagulation cascade, especially thrombin and fibrinogen, directly to the wound to bring about hemostasis. Bandaging of bleeding wounds is also a usual practice, in part to isolate and protect the wounded area, and also to provide a means to exert pressure on the wound, which can also assist in controlling bleeding.

While these methods may be carried out satisfactorily in cases of mild trauma or under conditions of “controlled” wounding (e.g. surgery), many situations in which such treatments are most needed are also those in which it is the most difficult to provide them. Examples of such wounds include, for example, those inflicted during combat, or unanticipated wounds that occur as the result of accidents. In such circumstances, survival of the wounded individual may depend on stopping blood loss from the wound and achieving hemostasis during the first few minutes after injury. Unfortunately, given the circumstances of such injuries, appropriate medical intervention may not be immediately available.

In particular, the treatment of penetrating wounds such as bullet wounds or some wounds from shrapnel is problematic. This is due to the difficulty in placing a bandage and/or therapeutic agents at the actual site of injury, which includes an area that is well below the body surface and difficult or impossible to access using conventional techniques.

Jiang et al. in Biomacromolecules, v. 5, p. 326-333 (2004) teaches electrospun dextran fibers. Agents associated with the fibers (e.g. BSA, lysozyme) are directly electrospun into the fibers. The fibers may also include other polymers electrospun with the dextran.

Bowlin et al., U.S. Patent Publication No. 2011/0150973, discloses a method of using hemostatic products on wounds. The method includes applying or delivering to a location of interest a hemostatic product. The hemostatic product includes electrospun dextran fibers that dissolve upon contact with liquid. The hemostatic product also includes one or more agents of interest associated with said electrospun dextran fibers. Applying or delivering results in dissolution of the electrospun dextran fibers in liquid at the location of interest to thereby release the one or more agents of interest into the liquid.

### SUMMARY OF THE INVENTION

An embodiment of the invention is directed to a surgical joint replacement kit that includes a first joint replacement component and a surgical sealant. The first joint replacement component is capable of being implanted to replace a joint between a first bone and a second bone. The first bone is cut in conjunction with implanting the first joint replacement component. Cutting the first bone causes a bodily fluid to flow from the first bone. The surgical sealant includes an electrospun dextran base and an effective amount of at least one of fibrinogen and thrombin. The at least one of fibrinogen and thrombin is applied to the electrospun dextran base to form the surgical sealant. The surgical sealant is capable of staunching the flow of the bodily fluid from the first bone.

Another embodiment of the invention is directed to a method of sealing tissue during a surgical procedure. A surgical sealant is provided that includes an electrospun dextran base and at least one of fibrinogen and thrombin. A bone is cut that causes a bodily fluid to flow from the cut bone. The surgical sealant is applied to the cut bone. The surgical sealant dissolves when contacted with the bodily fluid. The flow of the bodily fluid from the cut bone is staunched using the surgical sealant.

Another embodiment of the invention is directed to a method of sealing tissue during a total knee arthroplasty in a living body. A surgical sealant is provided that includes an electrospun dextran base and at least one of fibrinogen and thrombin. Tissue is cut to provide access to a femur and a tibia in the living body. The femur and the tibia are cut which causes blood to flow from the femur and the tibia. The surgical sealant is applied to the at least one of the femur and the tibia that is cut. The surgical sealant dissolves when contacted with the blood. The flow of the blood from the at least one of the femur and the tibia that is cut is staunched using the surgical sealant. A first joint replacement component is implanted adjacent to the femur. A second joint replacement component is implanted adjacent to the tibia. The cut tissue is closed in the absence of providing a port for drainage of fluids from the living body in which the total knee arthroplasty is performed.

Another embodiment of the invention is directed to a method of treating a burn on skin of a living body. A surgical sealant is provided that includes an electrospun dextran base and at least one of fibrinogen and thrombin. The surgical sealant is applied to a burned region on skin from which a bodily fluid is flowing. The surgical sealant dissolves when contacted with the bodily fluid. The flow of the bodily fluid from the burned region is staunched using the surgical sealant where it is not necessary to remove any portion of the surgical sealant that could damage the burned region or negatively impact healing of the burned region.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is a schematic view of a surgical joint replacement kit according to an embodiment of the invention.

### DETAILED DESCRIPTION OF THE INVENTION

An embodiment of the invention is directed to a method of reducing blood loss during total knee arthroplasty procedures using a sealant product. In certain embodiments, the sealant product comprises a plurality of layers.

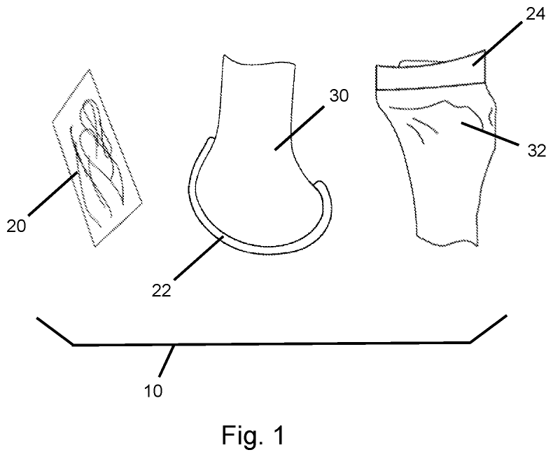

An embodiment of a surgical joint replacement kit is illustrated at 10 in FIG. 1. In certain configurations, the surgical joint replacement kit 10 may include a surgical sealant 20, a first joint resurfacing component 22 and a second joint resurfacing component 24.

While the surgical joint replacement kit 10 is illustrated as included two joint resurfacing components, the concepts of the invention may be adapted for alternative configurations such as including one joint resurfacing component or including at least three joint resurfacing components.

While forming the sealant product from a plurality of sheets of hemostatic material enhances the ability of the sealant product to provide the hemostatic functions, this structure presents challenges to maintaining the sheets in a desired position with respect to each other. The layers include a base to which at least one hemostatic agent is applied.

The base is fabricated from a material that substantially dissolves when coming into contact with a liquid. The material used to fabricate the base also does not cause any negative reactions when used in-vivo and is absorbed into the body such that it is not necessary to remove any portion of the base from the body.

In some embodiments of the invention, only spun dextran fibers are utilized in fabricating the base. The electrospun fibers are “dry” and should be protected from exposure to moisture to prevent premature dissolution. However, some water is associated with the fibers and fiber compositions can contain from about 7 to about 8 percent by weight water, but must be less than about 5 percent by weight when the fibers are sterilized by x-ray irradiation.

The products of the invention are usually formed of substantially homogeneous spun dextran. The amount of dextran per sealant product can vary widely, depending on the size of product that is being manufactured, with typical product formulations using from about 5-10 grams of dextran (usually 100,000-200,000 Mr) per sealant product.

However, the range can be extended widely, e.g. from as low as about 0.5 grams or less (for small sealant products) to as high as 100 or more grams per sealant product, for large products. In some embodiments of the invention, it may be helpful to use lesser amounts of dextran (e.g. about 0.1 to about 0.5 grams of dextran per product) to concentrate the active agents that are delivered by the product into a smaller volume.

Of more consequence is the concentration of dextran in the solution from which the fibers are spun. Generally, a solution of dextran for electrospinning will be of a concentration in the range of from about 0.1 to about 10 grams per ml of solvent, or from about 0.5 to about 5 grams per ml, and usually such a solution is at a concentration of about 1 gram per ml, ±about 0.15 mg. A preferred range would be from about 0.9 to about 1.1 grams of dextran per ml of solution that is to be spun.

The area (length and width) of the product of the invention can vary widely and can be adjusted by adjusting spinning parameters. In addition, the mats of dextran fibers can be cut to a desired size after spinning. Generally, the product will be from about 0.5 centimeters or less to about 30 centimeters or more in length and/or width, but larger or smaller sizes are also contemplated.

The height or thickness of the sealant product can likewise vary considerably depending on the intended use of the product. In certain embodiments, the product has a thickness of between about 1 millimeter and about 5 centimeters.

The thickness of the product (which is related to the volume) may impact the rate of dissolution of the dextran upon contact with liquid. For example, a thin product (e.g. about 2 millimeters), will dissolve more rapidly than a product that is thicker, providing the loft of the fibers is comparable.

In most embodiments, dissolution of the dextran fibers is extremely rapid, e.g. about 5 minutes or less after exposure to liquid, or about 4 minutes or less, or about 3 minutes or less, or about 2 minutes or less, or about 1 minute or less, e.g. the product typically takes only a few seconds to dissolve (e.g. from about 1 to about 20 seconds to dissolve).

This rapid dissolution may be referred to herein as “instantaneous” or “immediate” dissolution. Compression of an electrospun dextran mat may be used to modulate the rate of dissolution, with greater levels of compression inversely impacting the rate, i.e. generally, the greater the degree of compression, the slower the rate of dissolution. The rapid rate of dissolution is advantageous, particularly when delivering biologically active agents (e.g. hemostatic agents) to a site of action. Rapid dissolution of the carrier dextran fibers provides extremely rapid delivery of the hemostatic agents to the surgical incisions upon deployment of the sealant product.

Those of skill in the art will recognize that a plethora of liquid solvents exist in which it is possible to dissolve dextran. However, superior results for electrospinning dextran are generally achieved when the solvent is water, especially deionized or distilled or deionized, distilled (ddH2O) or other forms of relatively pure water. In addition, there is far less environmental impact associated with the use of water.

It has been found that, generally, high concentrations of salt in the solvent should be avoided. Whereas salt is often used to facilitate the spinning of some electrospun polymers, this is not the case for dextran. The concentration of salts in the spinning solution should be kept at a minimum to successfully form dextran fibers.

The one or more active agents that are associated with the dextran fibers of the product may be any active agent that it is desirable or advantageous to deliver to the site where the product is to be used or applied. In one embodiment of the invention, the product is used to deliver active agents, for example, to a location where a total knee arthroplasty is being performed.

Usually the active agents are bioactive agents that have a beneficial or therapeutic effect at the site of the total knee arthroplasty. In one embodiment, the total knee arthroplasty site is bleeding and it is desired to form a blood clot to stop or slow the bleeding. In this embodiment, the therapeutic substances of interest may include, for example, thrombin and fibrinogen, although other agents active in promoting hemostasis, including but not limited to capscian, may also be included.

In addition, electrospun or non-electrospun collagen, agents that absorb water, various dry salts that would tend to absorb fluids when placed in contact with e.g. blood; engineered thrombin or thrombin mimics; engineered fibrinogen; agents that cause vasospasm (e.g. ADP, 5-hydroxytryptamine, 5-HT and thromboxane, (TXA-2) to help contract and seal a bleeding vessel, etc. may also be included.

In addition, other components of the clotting cascade may be added to the sealant product. Examples of such additional components include tissue factors that are normally only expressed on the surface of damaged cells and which start the normal clotting cascade; serotonin which enhances platelet clumping and promotes vessel constriction; and other agents that are used to replace missing components of the clotting cascade in hemophilia, such as Factor 7 (which activates the so called external extrinsic coagulation cascade) and crude extracts of platelets. These agents essentially work to “jump start” clotting by initiating the cascade further down the reaction network. The various factors (and their alternative nomenclature and/or characteristics and/or activities) are as follows:

Factor VII (Proconvertin): serine protease, Vitamin K dependent synthesis in the liver;

Factor VIII: Glycoprotein binds vWF, produced by endothelium and liver;

Factor IX (Christmas-Eve Factor): serine protease;

Factor X (Stuart-Prowler Factor, Clotting Factor X): serine endopeptidase, converts prothrombin to thrombin; and

Factor XI (Plasma thromboplastin antecedent): serine protease, plasma protein;

Factor XII (Hageman factor): serine protease, plasma protein binds collagen;

Factor XIII (Fibrin stabilizing Enzyme): stabilizes fibrin polymer. plasma protein, also present in platelets and monocyte linage.

Additional active agents may also be used in the sealant product, examples of which include, for example, facilitating cell migration and remodeling. One or more of any of these active agents may be used in the practice of the present invention. The therapeutic agents must be amenable to drying and are associated with the other components of the sealant product in the dry state, since liquid may negatively affect at least one of the components used in the sealant product. For example, the active agents may be desiccated or lyophilized, or water may be removed by some other means.

Generally, the amount of water that is present in the substances when they are associated with the electrospun dextran fibers is less than about 5%, and preferably less that about 2%. These substances retain full or partial activity when rehydrated, e.g. in blood. Generally therapeutic substances associated with the sealant products of the invention retain, upon contact with liquid, at least about 25%, or about 50%, or even about 75 to 100% of their activity before drying or desiccation, as compared to standard preparations of the substance using standard assays that are known to those of skill in the art.

In some embodiments, thrombin or fibrinogen, or both, are associated with the sealant product. The at least one of the thrombin and the fibrinogen are used in an effective amount. As used herein, effective amount means that the concentration of the thrombin and/or fibrinogen is sufficient to staunch the flow of bodily fluid such as blood when the sealant product is brought into contact with the cut bone.

In some embodiments, at least one of the thrombin and the fibrinogen are obtained from a human source. In other embodiments, at least one of the thrombin and fibrinogen are salmon thrombin and fibrinogen. Advantages of using salmon as a source of these materials include but are not limited to the lack of concern about transmission of etiologic agents (e.g. viruses) that may occur when human and other mammalian sources of thrombin or fibrinogen (e.g. bovine) are used.

Salmon thrombin and fibrinogen are highly efficacious and have no deleterious side effects, when used in the pig model, which is a recognized animal model that is considered to be indicative of results in humans.

The quantity of fibrinogen added to the sealant product is generally in the range of from about 10 milligrams to about 3 grams. In certain embodiments, the amount of fibrinogen in each of the sealant products is between about 20 milligrams to about 1 gram.

The quantity of thrombin added to each of the sealant products is generally between about 10 and 10,000 NIH Units. In certain embodiments, the amount of thrombin in each of the sealant products is between about 20 and 6,000 NIH Units.

In some embodiments, the therapeutic agents may themselves be electrospun. For example, the therapeutic agents are dissolved in and spun from a solution. In some embodiments, the therapeutic agents may be electrospun into fibers. In other embodiments, the active agents may be electrospun into other forms such as droplets, beads, etc.

In some applications, active agents such as thrombin may be electrosprayed with sucrose to form sugar droplets, which tends to stabilize thrombin and can also “trap” other substances of interest for delivery to the sealant product.

For thrombin and fibrinogen, in most embodiments, these (or other) active agents are in a finely dispersed dry, particulate or granular form e.g. as a fine powder or dust, as electrospinning may tend to decrease their activity. In other words, the active agents are not electrospun either by themselves.

The provision of the substances in the form of a fine powder provides a large surface area of contact for dissolution when the materials come into contact with fluid. Generally, such particles will have average diameters of between about 1 and 10,000 microns, and, in certain embodiments, between about 10 and 1,000 microns.

Such dry solid particles may be formed by any of several means, including but not limited to grinding, pulverizing, crushing, etc. However, those of skill in the art will recognize that other forms of these active agents may also be included in the sealant product, e.g. flakes, films, sheets, strings, etc. Further, in some embodiments, thrombin and fibrinogen are in the form of electrospun droplets when associated with an excipient or carrier.

Association of substances of interest with the excipient or carrier may be accomplished by any of many suitable techniques that are known to those of skill in the art, and will depend in part on the precise form of the substance and the means at hand. For example, for powdered, particulate thrombin and fibrinogen, association may be carried out by sprinkling, shaking, blowing, etc. the agents onto a layer of the excipient or carrier.

Depending on the density of the fiber mat, the substances of interest may become relatively evenly dispersed throughout the woven mat of fibers or may be largely confined to the topmost section of the fiber mat. If no backing is present, the latter embodiment is preferable, to prevent the particulate substance of interest from falling through and out of the mat.

The density of the fibrous mat can be adjusted (e.g. increased), for example, by adjusting its thickness and/or by compressing the mat under pressure so that the fibers are closer together. Other techniques for association also exist, e.g. the placement of dry but liquid soluble sheets or strips of material onto or between layers of a carrier, electrospinning the added materials as a discrete layer or in discrete layers, etc., and any such technique may be employed.

The techniques for assembling the sealant products of the invention may be carried out manually or may be mechanized, or a combination of manual manipulation and mechanization may be used. For thrombin in particular, 5,000 NIH Units of thrombin is a relatively small volume of powder. Therefore, inert carriers or bulking agents such as dextrose may be added to insure more complete dispersal of active agents in the sealant product.

The association of substances of interest with the excipient may be carried out according to many different arrangements. For example, a first layer of excipient may be formed, and one or more of the substances may be associated with the first layer. Then another second layer of excipient may be formed on top of the substance(s) of interest, and the same or other substances of interest may be associated with the second layer, and so on.

A final or outermost layer of excipient may be added to prevent the dislodgement of substances of interest from the layer(s) below. The number of layers of excipient that are used in a sealant product of the invention may vary widely, from as few as 1-2 to as many as several dozen, or even several hundred, depending on the desired characteristics of the sealant product.

Typically, the sealant product will contain 1-2 layers. In other embodiments the sealant product may include between 2-20 layers. The very slight amount of moisture that is present in a prepared sealant product may help to trap and retain the thrombin and fibrinogen on the surface of the sealant product.

In some embodiments of the invention, the sealant products also include one or more support structures or support materials incorporated therein. For example, a backing may be incorporated into the sealant product. The support material may be used to apply the sealant product to the use location. In certain circumstances, the support material is removed or separated from the sealant product after the sealant product is applied.

The support material may be formed from various electrospun materials such as polyglycolic acid (PGA), polylactic acid (PLA), and their copolymers (PLGAs); charged nylon, etc. In one embodiment, the support material is compressed electrospun dextran fibers. By “compressed electrospun dextran fibers” we mean that electrospun dextran fibers are compressed together under pressure.

Compression of electrospun dextran fibers is carried out, for example, under pressure between two plates (e.g. a vice), and can compress a mat of fibers with a height (thickness) of about 3 inches to a sheet with a height of about 0.5 inches or even less (e.g. about 0.1 to about 0.4 inches). In some embodiments, the electrospun dextran fibers are electrospun directly onto a previously electrospun support material, while in other embodiments, the support material and the electrospun dextran fibers are associated after electrospinning of each, e.g. by joining of one or more layers of each.

In other embodiments, the support material is not an electrospun material but is some other (usually lightweight) material. Examples of such materials include but are not limited to gauze; various plastics; hydrogels and other absorbent materials that can facilitate absorption of blood and therefore clot formation; etc.

The support material may or may not be soluble in liquid, or may be slowly soluble in liquid, and may or may not be permeable to liquid. Slowly soluble materials include those from which absorbable or dissolving (biodegradable) stitches or sutures are formed, included PGA, polylactic and caprolactone polymers.

In certain embodiments, the support material may dissolve relatively quickly such as less than about 1 hour. In other embodiments, the support material may dissolve within from about 10 days to 8 weeks. In either case, the support material provides the advantage of not having to remove the sealant product and risk disrupting the clot.

However, in any case, the support material should not interfere with the immediate dissolution of the excipients and delivery of the active agents associated therewith into the liquid that dissolves the excipients. Thus, the support material might be only on one side of the sealant product, so that when the sealant product is, for example, a sealant product, and is applied to the use location, the sealant product is oriented so that the excipients come into direct contact with the blood and the support material does not, i.e. the support material is the “top” or outermost surface of the sealant product when used.

This arrangement could provide an advantage in that pressure could be applied to the use location through the support material, to facilitate the stoppage of bleeding. Alternatively, the support material may contain pores, openings or spaces that allow liquid to access the excipients in the sealant product even when the support material is present. For example, the support material may be a net or web of material that is insoluble (or slowly soluble) but that permits liquid to freely access the excipients and associated substances of interest.

One of skill in the art will be able to envision many other combinations and shapes of excipient layers and support materials that would provide advantages in particular scenarios. For example, excipients might be wrapped or wound around an elongated support such as a filament or string, or wrapped around a particular form with the shape of a cavity in which the sealant product is likely to be placed, such as a bullet hole, etc.

In another embodiment, each of the sheets is primarily fabricated from dextran and thrombin. Each of the layers may have a thickness of between about 0.001 inches and about 0.50 inches.

Depending on the type of location with which the sealant product is intended to be used, there may be between about 2 and 20 sheets placed in a stacked relationship to form the sealant product.

While it is possible to fabricate the sheets so that fibrinogen is also included in at least a portion of the sheets, in certain embodiments, fibrinogen is provided in a powder that is dispersed onto the surface of at least a portion of the sheets.

To enhance the ability to obtain optimal results from the sealant product, the layers used in fabricating the sealant product may be attached together in a manner that enables the layers to remain in a substantially stationary position with respect to each other.

Thereafter, the sheets are cut into a desired shape and size and then stacked upon each other. Once the desired number of sheets are stacked, the sheets are fused together to thereby prevent the sheets from moving with respect to each other. In one aspect of the invention, cutting causes the sheets to fuse together. Another suitable technique for fusing the layers together is ultrasonic welding. The ultrasonic welding avoids complications from the presence of adhesives.

Non-limiting examples of other techniques that may be utilized to fuse the layers in a substantially stationary position with respect to each other is sewing, crimping, heat sealing and applying a medical grade adhesive. Whatever technique is utilized to fuse the layers together, the technique should maintain the ability of the sealant product to substantially dissolve so that the patient does not experience complications from such undissolved components and so that the patient does not have to undergo additional surgical procedures to remove the undissolved components of the sealant product.

All such arrangements, shapes, and embodiments of carrier layers and support materials as described herein are intended to be encompassed by the invention.

The sealant product may be sterilized prior to use, generally by using electromagnetic radiation, for example, X-rays, gamma rays, ultraviolet light, etc. If thrombin is included in the sealant product, it may be desirable to reduce the moisture content of the sealant product (e.g. a bandage or gauze) to less than about 5%, to preserve thrombin activity during sterilization.

This moisture content reduction can be achieved by drying the fabricated sealant product, e.g., under a vacuum, or by using a fabrication method that reduces moisture content from the beginning. Typically, the sealant products are sterilized using X-rays in a dose of about 5 kilograys (kGray). Any method that does not destroy the carrier or the activity of substances associated with the fibers may be used to sterilize the sealant products of the invention.

In such cases, the sealant products may serve as a “scaffolding” or carrier for containing, storing and/or transporting the substance(s) until use, i.e. until contacted with liquid that dissolves the electrospun dextran fibers, concomitantly releasing the substances into the liquid. Such substances may include, for example, enzymes or their precursors (e.g. pro-enzymes or zymogens) and their substrates, substances that activate a protein or enzyme (e.g. proteases, cofactors, etc.), and the like.

The invention also relates to the use of stabilizers that resist the premature degradation of the components utilized in the sealant product. The stabilizer also enhances the usable shelf life of the sealant product. In certain embodiments, the stabilizer provides the sealant product with a shelf life of at least about 2 years. In other embodiments, the hemostatic sealant exhibits a shelf life of at least 3 years.

As used herein, the term usable shelf life means that the sealant product does not exhibit noticeable degradation when viewed without magnification or with magnification such as a magnifying glass or microscope.

One such stabilizer is adapted for use in conjunction with thrombin. It is believed that the thrombin stabilizer gets into the structure of the thrombin and thereby reduces the rate at which the thrombin breaks down. The at least one thrombin stabilizer may be mixed with the thrombin before the thrombin is mixed with the other components used to fabricate the sealant product.

In one embodiment, the thrombin stabilizer contains a sugar such as sucrose. In certain embodiments, the sucrose is used in the thrombin stabilizer at a concentration of up to about 5 percent by weight of the thrombin. In other embodiments, the sucrose concentration is about 1 percent by weight of the thrombin.

Prior to mixing the thrombin stabilizer with the thrombin, the thrombin stabilizer may be mixed with dextran. It is believed that the dextran enhances the ability of the sucrose to enter into the structure of the thrombin.

In certain embodiments, the dextran is used in the thrombin stabilizer at a concentration of up to about 5 percent by weight of the thrombin. In other embodiments, the dextran concentration is about 1 percent by weight of the thrombin.

Similarly, a stabilizer may be used in conjunction with the fibrinogen. Prior to applying the fibrinogen to the other components of the hemostatic bandage, the fibrinogen stabilizer may be mixed with the fibrinogen. It is believed that the fibrinogen stabilizer gets into the structure of the fibrinogen and thereby reduces the rate at which the fibrinogen breaks down.

In one embodiment, the fibrinogen stabilizer contains a sugar such as sucrose. In certain embodiments, the sucrose is used in the fibrinogen stabilizer at a concentration of up to about 5 percent by weight of the fibrinogen. In other embodiments, the sucrose concentration is between about 2 and 3 percent by weight of the fibrinogen. In still other embodiments, the sucrose concentration is about 1 percent by weight of the fibrinogen.

Prior to mixing the fibrinogen stabilizer with the fibrinogen, the fibrinogen stabilizer may be mixed with a solubility enhancing agent. It is believed that the solubility enhancing agent enhances the ability of the sucrose to enter into the structure of the fibrinogen. In certain embodiments, the solubility enhancing agent is a detergent. In other embodiments, the solubility enhancing agent is Pluronic.

In certain embodiments, the solubility enhancing agent is used in the fibrinogen stabilizer at a concentration of up to about 1 percent by weight of the fibrinogen. In other embodiments, the solubility enhancing agent concentration is about 0.002 percent by weight of the fibrinogen.

In another embodiment of the invention, the fibrinogen and thrombin are placed on the surface of and/or integrated into the matrix of a dissolving film. Using the fibrinogen and thrombin in such a configuration enables the sealant product to be positioned over the position on the person's body where the blood is being emitted and, as such, where hemostasis is desired.

The dissolvable film may be configured to dissolve relatively quickly when exposed to liquid such as blood. In certain embodiments, the film dissolves in less than about 30 seconds. In other embodiments, the film dissolves in less than about 5 seconds. An example of one suitable dissolving film is marketed by Hughes Medical Corp.

An example of another dissolving film is a dissolving paper that is fabricated from materials that do not pose a health hazard to the patient after the dissolving paper has dissolved. In certain embodiments, the dissolving paper may be fabricated from a material that enhances the ability of at least one of the fibrinogen and thrombin to achieve hemostasis. An example of one such dissolving paper is marketed by Daymark Technologies.

In another embodiment, the fibrinogen and thrombin are provided between two layers of a dissolvable material. An example of one such suitable dissolving film is marketed by Hughes Medical Corp. and which is discussed above.

The fibrinogen and thrombin may be provided in a variety of configurations using the concepts of the invention. In one such configuration, at least one of the fibrinogen and the thrombin are provided in a powder that is retained between the layers of the dissolvable material.

The dissolvable material should have sufficient structural integrity to retain the fibrinogen and thrombin therebetween while resisting interaction with the fibrinogen and thrombin. The dissolvable material should also dissolve relatively quickly when exposed to liquids such as blood such that the fibrinogen and thrombin are released therefrom.

As used herein, “quickly dissolving” means that the dissolvable material breaks down to a sufficient extent such that a significant portion of the fibrinogen and thrombin are in contact with the blood in less than about 30 seconds. In other embodiments, the dissolvable material breaks down in less than about 10 seconds.

The dissolvable material should also facilitate readily bonding such that two layers of the dissolvable material can be attached together around the edges thereof to thereby form an enclosure that is adapted to retain the fibrinogen and thrombin therein.

An example of one suitable technique for attaching the dissolvable materials to each other is applying a small amount of liquid to at least one of the pieces of material that are intended to be bonded together. The water causes a slight breakdown of the dissolvable materials such that when two layers of the dissolvable material are placed adjacent to each other, the layers of the dissolvable material bond together.

The dissolvable material may be fabricated from a variety of materials. The dissolvable material should not negatively impact the stability of the fibrinogen and thrombin. The material used to fabricate the dissolvable layer should also be selected to not have any adverse health effects on the person or animal on which the product is intended to be used.

In certain embodiments, the material used to fabricate the sealant product may alone or in conjunction with the fibrinogen or thrombin enhance the rate of hemostasis. Examples of components that may be used for the dissolvable material include cellulose-derived materials.

In an alternative configuration, the enclosure may be configured to breakdown over an extended period of time. As the enclosure breaks down, the fibrinogen and thrombin may be discharged from the sealant product.

By controlling the rate at which the fibrinogen and thrombin are discharged from the sealant product and/or the rate at which the enclosure degrades, the sealant product minimizes the formation of a clot having a relatively large size but rather may facilitate the formation of a plurality of clots having a smaller size. Such smaller clots may be more readily broken down within the body than if relatively large clots were caused to be formed.

In another embodiment, the fibrinogen and thrombin are compressed into a tablet. In addition to the fibrinogen and thrombin, the tablet may also include at least one excipient. The excipient should facilitate not only holding together the fibrinogen and thrombin as well as promoting relatively quickly dissolving of the tablet.

As used herein, the term “relatively quickly” means that the tablets dissolve when placed in a liquid in less than about 30 seconds. In other configurations, the tablets dissolve in less than about 10 seconds. Quickly dissolving the tablets enables the fibrinogen and the thrombin to be quickly released from the tablets such that these materials may provide rapid hemostasis.

Additionally, in certain embodiments, the excipients that are used in formulating the tablets should not decrease the stability and/or solubility of the fibrinogen and the thrombin. In certain embodiments, the excipients used in formulating the tablets should increase the stability of the fibrinogen and thrombin.

An example of one such excipient is sorbitol, which has been formed into small particles such as by using spray-drying. In one such configuration, the particles have a generally spherical shape and have a generally uniform size.

The spray-dried sorbitol particles not only provide advantageous flowability characteristics but also exhibit desirable compactability characteristics when forming the tablets using a direct compression technique.

Additionally, the spray-dried sorbitol particles provide good solubility for release of the fibrinogen and thrombin from the tablets. An example of one such spray-dried sorbitol particle is marketed by SPI Pharma under the designation SORBITAB SD 250.

Another excipient that may be used in fabricating the tablets is mannitol, which has been formed into small particles such as by using spray drying. The particles may be formed with a narrow particle size distribution, which reduces the potential of the components segregating while the tablets are being formed.

An advantage of the mannitol is that this material is non-hydroscopic such that the mannitol does not add moisture to the other components used in the tablets or contribute to moisture pickup either during the process of forming the tablets or after the tablets have been formed. The mannitol thereby protects the water-sensitive fibrinogen and thrombin.

The spray-dried mannitol particles not only provide advantageous flowability characteristics but also exhibit desirable compactability characteristics when forming the tablets using a direct compression technique.

The spray-dried mannitol particles also promote rapid disintegration or dissolvability of the tablets such that the fibrinogen and thrombin can be quickly released from the tablets. An example of one such spray-dried mannitol particle is marketed by SPI Pharma under the designation MANNOGEM EZ.

Other materials that may be used as excipients when preparing the tablets include fructose and maltose. Similar to the other excipients that are discussed above, the preceding excipients may be formed into small particles before being mixed with the other components that are used in the tablets.

Another excipient that may be used in conjunction with fibrinogen and thrombin is a quick dissolving platform that is marketed under the designation PHARMABURST 500 by SPI Pharma. This material provides the tablets with the ability to be rapidly dissolved while also providing desirable characteristics for compaction and friability.

Depending on the excipient that is used in the tablet, it may also be desirable to use a lubricant when preparing the tablet. The lubricant may enhance the physical properties of the tablets. Examples of such physical properties include brittleness, friability and hardness. An example of one such lubricant is sodium stearyl fumarate, which is available from SPI Pharma under the designation LUBRIPHARM.

The concentration of the lubricant that is used in fabricating the tablets may depend on a variety of factors such as the types of excipients that are used. In certain embodiments, the concentration of the lubricant is up to about 5 percent by weight. In other embodiments, the concentration of the lubricant is between about 2 and 3 percent by weight. In still other embodiments, the concentration of the lubricant is about 2.5 percent by weight.

Once the components are mixed together, the mixture is subjected to compression, which thereby causes the components to form the tablets. In certain embodiments, the compressive force is at least 5,000 psi. In other embodiments, the compressive force is between about 10,000 psi and about 12,000 psi.

When preparing the tablets using the preceding process, it may not be necessary to include dextran. Even though dextran may not be required, it is possible to use dextran along with the other components that are used to formulate the tablets.

In another embodiment of the invention, the fibrinogen and thrombin may be incorporated into a fast dissolving tablet such as by using technology marketed by Catalent Corporation under the designation Zydis.

The fast dissolving tablets dissolve in less than 30 seconds and, in some configurations, dissolve in less than about 5 seconds. Quickly dissolving the tablets is important because as the tablets dissolve, the fibrinogen and thrombin contained therein is released and can thereby produce hemostasis.

The amount of the fibrinogen and thrombin used in the tablet may be selected based upon the volume of bleeding. In certain embodiments, there is up to about 1 gram of fibrinogen and thrombin in each of the tablets. In other embodiments, there is about 500 micrograms of fibrinogen and thrombin in each of the tablets.

In another embodiment of the invention, the fibrinogen and thrombin are incorporated into foam. An example of one such suitable foam is an absorbable gelatin sponge such as is available under the designation VETSPON from Novartis.

Depending on the application at which it is desired to use the hemostatic sponge, it may be desirable to prewet the hemostatic sponge prior to the hemostatic sponge being applied to the region where hemostasis is desired.

Another advantage of using the foam is that the foam may be configured to be bendable so that the hemostatic foam can be bent into a configuration that conforms to the shape of the region in which the hemostasis is desired. Once the hemostatic foam is bent into the desired configuration, it may remain in that configuration even without a fastening device being used to hold the hemostatic foam in the desired shape and/or position.

Similar to the foam that is described above, the foam may be either open cell foam or closed cell foam. The foam should not have a strong affinity for either fibrinogen or thrombin so that when the fibrinogen and thrombin are exposed to water, these components are released from the foam.

The fibrinogen and thrombin may be incorporated into the components that are used to fabricate the foam such that rather than the fibrinogen and thrombin being applied to a surface of the foam, the fibrinogen and thrombin are dispersed through the matrix of the foam.

Such a configuration facilitates ongoing release of the fibrinogen and thrombin from the foam and may be particularly beneficial when it is desired to form a clot in a region of the body that is likely to experience rebleeding.

In addition to being used to produce hemostasis in humans, the concepts of the invention may be adapted for use in conjunction with other animals. Examples of such animals on which the invention can be used include dogs and cats.

In another embodiment of the invention, an effective amount of water is mixed with dextran to form an aqueous dextran solution. Thereafter, the aqueous dextran solution is electrospun to form a dextran sheet.

The dextran sheet may be stored until it is desired to fabricate the sealant product. In one such configuration, the dextran sheet is rolled. Rolling of the dextran sheet not only reduces the area taken up by the dextran sheet while the dextran sheet is being stored but also reduces the potential that the dextran sheet will be damaged prior to fabricating the sealant product.

When the dextran sheet is being rolled, care should be exercised to not roll the dextran sheet too tightly because such a process would increase the density of the dextran sheet. Alternatively, tightly rolling the dextran sheet may be desired to increase the density of the dextran sheet prior to fabricating the sealant product.

The thrombin and fibrinogen are mixed together at the ratio discussed in the other portions of this patent application just before it is desired to fabricate the sealant product. The mixing should provide a relatively uniform dispersion of the thrombin and fibrinogen in the mixture.

In certain embodiments, the thrombin is dispersed on the dextran sheet to provide a thrombin concentration of between about 2 and 200 NIH Units of thrombin per square centimeter of the dextran sheet.

In certain embodiments, the fibrinogen is dispersed on the dextran sheet to provide a fibrinogen concentration of between about 20 and 60 grams of fibrinogen per square centimeter of the dextran sheet.

The dextran sheet is unrolled and the thrombin and fibrinogen mixture is dispersed over the surface of the dextran sheet. In certain embodiments, the thrombin and fibrinogen mixture is dispersed in a substantially uniform manner over the surface of the dextran sheet. This even dispersion is desired because it enables each portion of the sealant product to have a substantially hemostatic activity.

This process is repeated and the sheets are stacked until the sealant product exhibits a desired amount of hemostatic activity. In certain embodiments, the sealant product includes between about 2 and 20 dextran layers.

The thrombin and fibrinogen mixture is not placed on the uppermost layer of the dextran sheet. Using this configuration, the thrombin and fibrinogen are located at an interior location in the sealant product. Fabricating the sealant product in this manner enhances the ability to retain thrombin and fibrinogen inside of the sealant product even though the thrombin and fibrinogen are sprinkled on the surface of the dextran sheet.

While it is possible to put thrombin and fibrinogen on the outside of the sealant product, a portion of the thrombin and fibrinogen may become dissociated from the sealant product prior to use. In view of the cost of thrombin and fibrinogen, it is desirable for substantially all of the thrombin and fibrinogen to remain associated with the sealant product until it is desired to use the sealant product to maximize the efficacy of the sealant product.

Even though the thrombin and fibrinogen are mixed together prior to placing the thrombin and fibrinogen on the dextran sheet, the thrombin and fibrinogen are sufficiently dispersed on the dextran sheet so that the thrombin and fibrinogen do not react prior to placing the sealant product at the location where hemostasis is desired.

The sealant product is then cut into pieces. In certain embodiments, the pieces may be formed in a generally square shape. The size of the pieces may be selected based upon the intended use of the sealant product. For example, when the sealant product is intended for surgical applications, the pieces may have a smaller size than if the sealant product is intended for trauma applications.

A cutter may be used to cut the sealant product into the desired size. In addition to cutting the sealant product into pieces, the cutter may cause the layers of the dextran sheets that are adjacent to the cutter to be compressed together. This compression causes the dextran layers to stay together.

In certain embodiments, the pieces of the sealant product are vacuum packaged. In addition to maintaining the sealant product sterile, the vacuum packaging also compresses the layers in the sealant product, which enhances the ability of the layers to resist separation after the sealant product is removed from the package prior to use.

This process thereby enhances the ability to use the sealant product because the layers in the sealant product resist coming apart. An advantage of using this process is that no additional steps are necessary to retain the layers together. Additionally, it does not require the use of additional components and/or additional processing steps, which could affect the efficacy of the sealant product.

Blood loss is a significant issue that must be addressed in patients who are undergoing total knee arthroplasty because of blood loss during the surgery and after the surgery. This blood loss and the burden of an associated blood transfusion present significant morbidity issues for the patient. Additionally, the increased blood loss can delay patient rehabilitation and prolong hospitalization. Other bodily fluids can also be lost as a result of a bone being cut. An example of another bodily fluid is bone marrow.

In this regard, there currently is a focus on surgical instrumentation and techniques that minimize perioperative blood loss and, as such, lower transfusion rates. Notwithstanding the desire to reduce blood loss, it is relatively common to utilize a drain in conjunction with total knee arthroplasty to reduce the potential of postoperative hematomas and reduce the potential of infection.

On technique that is typically used to reduce blood flow to the region where the total knee arthroplasty is being performed is application of a tourniquet. The reduced bleeding is also beneficial to the surgeon by enhancing visualization of the operative field and enhancing the ability to cement the implant components to the bones.

After access to the knee joint is attained, the arthritically damaged areas at the bottom of the femur and the top of the tibia are removed such as cutting with a bone saw. The cut ends of the femur and/or the tibia are reshaped to substantially conform to the shape of a surface of the prosthesis that is to be placed adjacent to the bone using conventionally known techniques.

FIG. 1 illustrates the first joint replacement component 22 placed over the femur 30 and the second joint component 24 placed over the tibia 32 where the end of the femur 30 has not been cut and the end of the tibia 32 has been cut to change the shape thereof. In such a configuration, the first joint replacement component 22 is formed with a shape that generally conforms to a shape of the end of the femur 30 over which the first joint replacement component 22 is placed. An end of the tibia 32 is cut off so that the cut end of the tibia 32 has a shape that is similar to the surface of the second joint replacement component 24 that is adjacent to the cut end of the tibia 32. A person of skill in the art will appreciate that the bone that is cut may be switch or that both the femur 30 and the tibia 32 may be cut.

The sealant product is applied to the cut ends of the femur and/or the tibia after the damaged portions of the bones are removed and/or the bones are reshaped to substantially conform to the prosthesis.

Application of the sealant product to the cut bones results in dissolution of the dextran fibers in blood, which in turn releases the active agents. Thrombin and fibrinogen that are associated with the hemostatic product are in forms that are biologically active when they come into contact with blood. Hence, upon dissolution, the thrombin acts on the fibrinogen, converting it to fibrin, which then forms a clot and thereby staunch the flow of blood.

A similar procedure can be used in conjunction with a total hip arthroplasty because cutting of the bones in such a procedure results in a similar blood loss that must be controlled to protect the patient as well as to provide the surgeon with the ability to clearly visualize the bones that are being cut and/or reshaped.

Yet another area in which the invention can be used is in treating burns. A significant challenge with burn patients is stopping bleeding because the burned tissue is very fragile. Because the sealant product of this invention dissolves upon contact with blood, the sealant product can be applied to bleeding tissue.

The fact that the sealant product fully dissolves means that it is not necessary to remove any part of the sealant product after it has been applied to the burned tissue which thereby avoids further damage to the burned tissue and/or reinitiation of bleeding during the removal process. The sealant product can also include components that are beneficial in treating burns such as encouraging regrowth of skin and preventing infection.

Yet another application for the sealant product is in conjunction with treating cancer patients during surgery to remove tissue. Generally, when a tumor is removed from the human body, some tissue needs to be cut. The bleeding associated with this tissue needs to be stopped. Additionally, it is often desirable to provide a pharmaceutical that acts on cancer cells that are not removed from the body.

Bleeding that is occurring proximate to where the cancer cells were removed needs to be stopped and contact between the sealant product and the blood causes the sealant product to dissolve and such dissolving releases the active components, which cause hemostasis.

The dissolving of the sealant product also releases the pharmaceuticals that act on the cancer. As described above, it is possible to control the rate at which the sealant product dissolves such as by compressing the sealant product and/or by compressing the electrospun dextran base.

This controlled dissolving may be used to control the release of the pharmaceuticals and/or other active agents from the sealant product. The sealant product may be formed with areas having different rates of dissolution such that the pharmaceuticals and/or other active agents are released from the sealant product over an extended period of time.

In the preceding detailed description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The preceding detailed description, therefore, is not to be taken in a limiting sense, and the scope of the present invention is defined by the appended claims.

It is contemplated that features disclosed in this application, as well as those described in the above applications incorporated by reference, can be mixed and matched to suit particular circumstances. Various other modifications and changes will be apparent to those of ordinary skill.

1. A surgical joint replacement kit comprising:
a first joint replacement component that is capable of being implanted to replace a joint between a first bone and a second bone, wherein the first bone is cut in conjunction with implanting the first joint replacement component and wherein cutting the first bone causes a bodily fluid to flow from the first bone; and
a surgical sealant comprising:
an electrospun dextran base; and
an effective amount of at least one of fibrinogen and thrombin that is applied to the electrospun dextran base to form the surgical sealant, wherein the surgical sealant is capable of staunching the flow of the bodily fluid from the first bone.

2. The surgical joint replacement kit of claim 1, and further comprising a second joint replacement component, wherein the second bone is cut in conjunction with implanting the second joint replacement component, wherein cutting the second bone causes a bodily fluid to flow from the second bone, wherein the surgical sealant is capable of staunching the flow of the bodily fluid from the second bone, wherein the second joint replacement component is operably movable with respect to the first surgical joint replacement component after the first joint replacement component and the second joint replacement component are implanted.

3. The surgical joint replacement kit of claim 1, wherein the surgical sealant comprises a plurality of layers and each of the layers comprises one of the electrospun dextran base and wherein the layers are joined together so that the layers remain in a substantially stationary position with respect to each other prior to use of the surgical sealant.

4. The surgical sealant of claim 1, and further comprising a stabilizer that is utilized in conjunction with at least one of the electrospun dextran base, the fibrinogen and the thrombin.

5. A method of sealing tissue during a surgical procedure comprising:
providing a surgical sealant comprising an electrospun dextran base and at least one of fibrinogen and thrombin;
cutting a bone, which causes a bodily fluid to flow from the cut bone;
applying the surgical sealant to the cut bone;
dissolving the surgical sealant when contacted with the bodily fluid; and
staunching the flow of the bodily fluid from the cut bone using the surgical sealant.

6. The method of claim 5, and further comprising:
prior to cutting the bone, cutting tissue to provide access to the bone; and
closing the cut tissue after completion of the surgical procedure associated with the cut bone in the absence of providing a port for bodily fluids to drain from a body in which the bone is located.

7. The method of claim 6, wherein dissolving the surgical sealant comprises dissolving of the electrospun dextran base and wherein the dissolved electrospun dextran is absorbed into the body in which the surgical sealant is used so that none of the surgical sealant needs to be removed from the body prior to closing the cut tissue.

8. The method of claim 6, wherein cutting the bone causes the bone to at least partially conform to a joint replacement component, and wherein the method further comprises:
prior to closing the cut tissue, implanting the joint replacement component adjacent to the cut bone.

9. The method of claim 8, wherein staunching the flow of bodily fluid from the cut bone facilitates cutting the bone to at least partially conform to the joint replacement component.

10. The method of claim 5, wherein staunching the flow of bodily fluid from the cut bone obviates providing a blood transfusion in conjunction with the surgical procedure.

11. The method of claim 5, wherein the surgical sealant rapidly dissolves upon contact with the bodily fluid and wherein the bodily fluid is blood.

12. The method of claim 5, wherein the surgical procedure is a total knee arthroplasty and wherein cutting the bone comprises cutting at least one of the tibia and the femur.

13. The method of claim 5, wherein the surgical sealant comprises a plurality of layers and each of the layers comprises one of the electrospun dextran base.

14. A method of performing a total knee arthroplasty in a living body, wherein the method comprises:
providing a surgical sealant comprising an electrospun dextran base and at least one of fibrinogen and thrombin;
cutting tissue to provide access to a tibia and a femur in the living body;
cutting at least one of the tibia and the femur, which causes blood to flow from the at least one of the tibia and the femur that is cut;
applying the surgical sealant to the at least one of the tibia and the femur that is cut;
dissolving the surgical sealant when contacted with the blood;
staunching the flow of the blood from the at least one of the tibia and the femur that is cut using the surgical sealant;
implanting a first joint replacement component adjacent to the tibia;
implanting a second joint replacement component adjacent to the femur; and
closing the cut tissue in the absence of providing a port for drainage of fluids from the living body in which the total knee arthroplasty is performed.

15. The method of claim 14, wherein dissolving the surgical sealant comprises dissolving of the electrospun dextran base and wherein the dissolved electrospun dextran is absorbed into the living body in which the surgical sealant is used so that none of the surgical sealant needs to be removed from the living body prior to closing the cut tissue.

16. The method of claim 14, wherein cutting the tibia causes the tibia to at least partially conform to the first joint replacement component and wherein cutting the femur causes the femur to at least partially conform to the second joint replacement component.

17. The method of claim 14, wherein staunching the flow of blood from the tibia facilitates cutting the tibia to at least partially conform to the first joint replacement component and wherein staunching the flow of blood from the femur facilitates cutting the femur to at least partially conform to the second joint replacement component.

18. The method of claim 14, wherein staunching the flow of blood from the at least one of the tibia and the femur that is cut obviates providing a blood transfusion in conjunction with the surgical procedure.

19. The method of claim 14, wherein the surgical sealant comprises a plurality of layers and each of the layers comprises one of the electrospun dextran base.

20. A method of treating a burn on skin of a living body comprising:
providing a surgical sealant comprising an electrospun dextran base and at least one of fibrinogen and thrombin;
applying the surgical sealant to a burned region on skin from which a bodily fluid is flowing;
dissolving the surgical sealant when contacted with the bodily fluid; and
staunching the flow of the bodily fluid from the burned region using the surgical sealant where it is not necessary to remove any of the surgical sealant that could damage the burned region or negatively impact healing of the burned region.

21. The method of claim 20, wherein the surgical sealant further comprises at least one of a component for encouraging regrowth of skin and a component for reducing potential of infection.

22. The method of claim 20, wherein the surgical sealant comprises a plurality of layers and each of the layers comprises one of the electrospun dextran base.
